(19) United States
(12) Patent Application Publication
(10) Pub. No.: US 2026/0069568 A1
(43) Pub. Date: Mar. 12, 2026

(54) GPR146 MODULATOR AND METHODS THEREOF

(71) Applicant: National University of Singapore, Singapore (SG)

(72) Inventor: Haojie YU, Singapore (SG)

(21) Appl. No.: 19/324,337

(22) Filed: Sep. 10, 2025

(30) Foreign Application Priority Data
Sep. 10, 2024 (SG) 10202402809P

(51) Int. Cl. A61K 31/407; A61P 3/06

(52) U.S. Cl. CPC A61K 31/407; A61P 3/06

(57) ABSTRACT

The present disclosure concerns a method of treating a disease and/or condition associated with GPR146 in a subject in need thereof, comprising administering to the subject a therapeutically effective amount of a compound of Formula (I) or a pharmaceutically acceptable salt, solvate, stereoisomer or prodrug thereof:

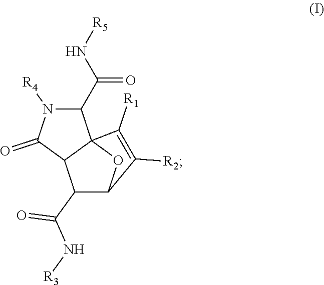

wherein R₁ is selected from H, halo, cyano, optionally substituted alkyl, and optionally substituted alkenyl; R₂ is selected from H, halo, cyano, optionally substituted alkyl, and optionally substituted alkenyl; R₃ is selected from optionally substituted aryl and optionally substituted heteroaryl; R₄ is selected from optionally substituted alkyl and optionally substituted alkenyl; R₅ is selected from optionally substituted cycloalkyl and optionally substituted heterocyclyl. The present disclosure also concerns a pharmaceutical composition comprising a compound of Formula (I).

### CROSS-REFERENCE TO RELATED APPLICATIONS

This application claims the benefit of the priority to Singaporean patent application number 10202402809P, filed on Sep. 10, 2024, the disclosure of which is incorporated by reference herein.

### TECHNICAL FIELD

The present invention relates, in general terms, to GPR146 modulator and their methods of use thereof.

### BACKGROUND

G-protein-coupled receptors (GPCRs) are targets for nearly 40% of all prescription drugs on the market and orphan GPCRs currently have become a primary focus for drug development. Orphan GPR146 is a promising drug target for hyperlipidedia, coronary artery disease, liver inflammation, obesity, and hypertension. Human genetic studies have uncovered that GPR146 is significantly associated with many cardio-metabolic diseases traits, including blood total cholesterol, LDL cholesterol, TG levels; liver inflammation enzyme (AST, GGT, ALP) levels; and hypertension. GPR146 also regulates hypercholesterolemia and liver inflammation in mouse models. Recently, studies from other groups suggested that GPR146 regulates pulmonary hypertension. This suggests that GPR146 inhibition may be an effective strategy in reducing blood LDL-cholesterol, TG levels, obesity, liver inflammation, and hypertension. Hence, developing small molecules to inhibit GPR146 may potentially be an effective strategy to treat these diseases associated with GPR146.

There are currently no FDA-approved therapies that target GPR146. Current drug candidates for GPR146 are antibodies, oligonucleotides, ligand fragments, and proteins. Such drug candidates often involves complicated and lengthy synthesis and purifications steps, which results in high cost. There is a need for small molecule drug compounds.

It would be desirable to overcome or ameliorate at least one of the above described problems.

### SUMMARY

The present disclosure concerns a method of treating a disease and/or condition associated with GPR146 in a subject in need thereof, comprising administering to the subject a therapeutically effective amount of a compound of Formula (I) or a pharmaceutically acceptable salt, solvate, stereoisomer or prodrug thereof:

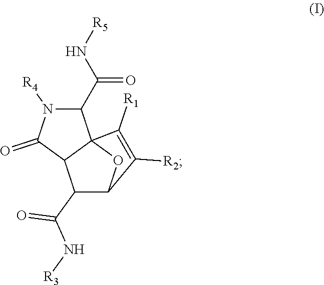

wherein
R₁ is selected from H, halo, cyano, optionally substituted alkyl, and optionally substituted alkenyl;
R₂ is selected from H, halo, cyano, optionally substituted alkyl, and optionally substituted alkenyl;
R₃ is selected from optionally substituted aryl and optionally substituted heteroaryl;
R₄ is selected from optionally substituted alkyl and optionally substituted alkenyl; and
R₅ is selected from optionally substituted cycloalkyl and optionally substituted heterocyclyl.

In some embodiments, R₁ is H.

In some embodiments, R₂ is H.

In some embodiments, R₃ is aryl optionally substituted with alkoxy. In some embodiments, R₃ is aryl meta substituted with alkoxy.

In some embodiments, R₄ is selected from C₁-C₅ alkyl substituted with optionally substituted aryl and C₁-C₅ alkyl substituted with optionally substituted cycloalkenyl. In some embodiments, R₄ is C₁-C₅ alkyl substituted with ortho substituted aryl. In some embodiments, R₄ is C₁-C₅ alkyl substituted with cyclohexenyl.

In some embodiments, R₅ is optionally substituted cyclohexyl. In some embodiments, R₅ is cyclohexyl optionally substituted with alkyl. In some embodiments, R₅ is cyclohexyl ortho substituted with alkyl.

In some embodiments, the compound of Formula (I) is a compound of Formula (Ia):

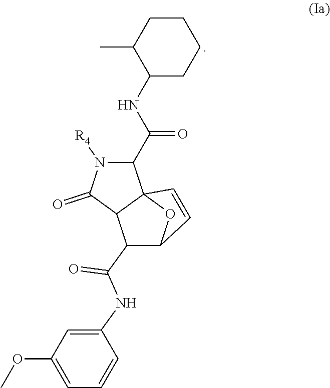

In some embodiments, the compound of Formula (I) is selected from:

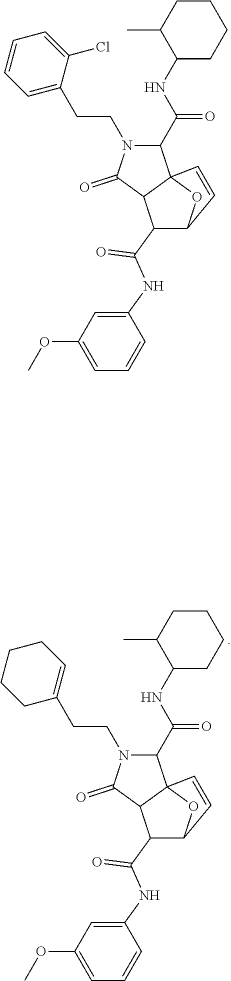 [SMILES: COc1cccc(NC(=O)C2C3C=CC4(O3)C2C(=O)N(CCc2ccccc2Cl)C4C(=O)NC2CCCCC2C)c1 ; COc1cccc(NC(=O)C2C3C=CC4(O3)C2C(=O)N(CCC2=CCCCC2)C4C(=O)NC2CCCCC2C)c1]

The present disclosure concerns a method of treating a disease and/or condition associated with GPR146 in a subject in need thereof, comprising administering to the subject a therapeutically effective amount of a compound of Formula (Ia) or a pharmaceutically acceptable salt, solvate, stereoisomer or prodrug thereof:

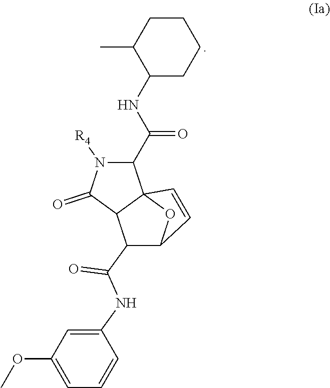

In some embodiments, the disease and/or condition associated with GPR146 is a disease or condition associated with a dysregulation of GPR146.

In some embodiments, the disease and/or condition associated with GPR146 is a cardiometabolic disease.

In some embodiments, the disease and/or condition associated with GPR146 is selected from cardiovascular disease, diabetes, kidney disease, liver disease, and a combination thereof.

In some embodiments, the disease and/or condition associated with GPR146 is selected from hyperlipidedia, hypercholesterolemia, hypertriglyceridemia, atherosclerosis, obesity, fatty liver disease, coronary artery disease, liver inflammation, hypertension, and a combination thereof.

In some embodiments, the compound of Formula (I) inhibits GPR146.

In some embodiments, the compound of Formula (I) inhibits extracellular signal-regulated kinase (ERK) activation.

In some embodiments, the compound of Formula (I) is characterised by a IC50 of about 1.5 μM to about 3.5 μM. In some embodiments, the compound of Formula (I) is characterised by a IC50 of about 2.5 μM.

In some embodiments, the compound of Formula (I) is characterised by a reduction in blood total cholesterol level of about 30% to about 60%. In some embodiments, the reduction is about 50%.

In some embodiments, the compound of Formula (I) is characterised by a reduction in triglyceride levels of about 15% to about 25%. In some embodiments, the reduction is about 21%.

In some embodiments, the compound of Formula (I) is characterised by a cytotoxicity of about 50% to about 95%.

The present disclosure also provides a pharmaceutical composition comprising a compound of Formula (I), a compound of Formula (Ia) or a pharmaceutically acceptable salt, solvate or prodrug thereof.

### BRIEF DESCRIPTION OF THE DRAWINGS

Embodiments of the present invention will now be described, by way of non-limiting example, with reference to the drawings in which:

FIG. 1 shows knockout of GPR146 protecting against Hypercholesterolemia and Atherosclerosis in LDLR-deficient mice. (A) Representative images of plasma (top layer) isolated from Gpr146 wild-type (Gpr146+/+) and knockout (Gpr146−/−) male mice lacking LDLR fed WD for 16 weeks. (B) and (C) Plasma TC (B) and TG (C) levels of Gpr146+/+ and Gpr146−/− littermates lacking LDLR fed WD for 16 weeks. (n=6-10 mice per group, by Student's t test). (D) Representative images of aortas before and after Oil Red O staining in Gpr146+/+ and Gpr146−/− mice lacking LDLR fed WD for 16 weeks. (E) Quantification of aortic lesion areas (expressed as a percentage of the lumen area in full-length aorta) in male mice lacking LDLR (n=6-10 mice per group, by Student's t test). *p<0.05, **p<0.01; bars in B, C and E indicate mean ±s.d.

FIG. 2 shows knockdown of GPR146 by AAV-Delivered shRNA lowering plasma lipid levels in mice lacking LDLR (A and B). Plasma TC (A) and TG (B) levels of male mice lacking LDLR 6 weeks after injection of AAV8-scramble control or AAV8-Gpr146-shRNA viruses (n=5-6 mice per group, by Student's t test). *p<0.05, **p<0.01; bars in A-D indicate mean ±s.d.

FIG. 3 shows Gpr146 regulates diet-induced obesity in mouse. (A) Body weight of male Gpr146+/+ and Gpr146−/− littermates fed chow or HFD as indicated (n=10-15 mice in each group). (B) MRI analysis of fat mass of male Gpr146+/+ and Gpr146−/− littermates fed HFD for 3 months (n=8 mice in each group). (C) Representative images (C) of eWAT and ingWAT from male Gpr146+/+ and Gpr146−/− littermates fed HFD for 3 months (n=8 mice per group). (D) Hepatic Triglyceride content in mice upon chow or HFD feeding for 3 months (n=8-13 mice per group).*p<0.05, **p<0.01; bars in A indicate mean±s.e.m., bars in B-D indicate mean±s.d.

FIG. 4 shows a schematic diagram showing the crude ligand solution preparation pipeline.

FIG. 5 shows an embodiment of a compound of Formula (I) as an antagonist of GPR146. (A) High-throughput compound screening identifies 27 GPR146-specific antagonist candidates. (B) Structure of an embodiment of a compound of Formula (I). (C) Dose effect of an embodiment of a compound of Formula (I) on inhibiting GPR146 activity through β-arrestin recruitment assay. (D) Western blot of phosphorylated ERK1/2 (PERK) and total ERK1/2 in 293 FT cells with overexpression of GFP or human GPR146. (E) and F) Plasma TG (E) and TC (F) of chow-fed female Ldlr−/− mice treated with vehicle or an embodiment of a compound of Formula (I) for the period of time indicated (n=3 mice per group, by Student's t test). *p<0.05, **p<0.01; bars in (E) and (F) indicate mean±s.d.

### DETAILED DESCRIPTION

“Alkyl” refers to monovalent alkyl groups which may be straight chained or branched and preferably have from 1 to 10 carbon atoms or more preferably 1 to 6 carbon atoms. Examples of such alkyl groups include methyl, ethyl, n-propyl, iso-propyl, n-butyl, iso-butyl, n-hexyl, and the like.

“Alkoxy” refers to the group alkyl-O— where the alkyl group is as described above. Examples include, methoxy, ethoxy, n-propoxy, iso-propoxy, n-butoxy, tert-butoxy, sec-butoxy, n-pentoxy, n-hexoxy, 1,2-dimethylbutoxy, and the like.

“Alkenyl” refers to a monovalent group having at least one carbon-carbon double bond which may be straight chain or branched and preferably have from 1 to 10 carbon atoms. Examples include ethenyl, propenyl, butenyl, I-methyl-2-buten-I-yl, heptenyl, octenyl, and the like.

“Halo” or “halogen” refers to fluoro, chloro, bromo and iodo.

“Aryl” refers to an unsaturated aromatic carbocyclic group having a single ring (e.g. phenyl) or multiple condensed rings (e.g. naphthyl or anthryl), preferably having from 6 to 14 carbon atoms. Examples of aryl groups include phenyl, naphthyl and the like.

“Heteroaryl” refers to a monovalent aromatic heterocyclic group which fulfils the Hückel criteria for aromaticity (i.e. contains 4n+2 n electrons) and preferably has from 2 to 10 carbon atoms and 1 to 4 heteroatoms selected from oxygen, nitrogen, selenium, and sulfur within the ring (and includes oxides of sulfur, selenium and nitrogen). Such heteroaryl groups can have a single ring (e.g. pyridyl, pyrrolyl or N-oxides thereof or furyl) or multiple condensed rings (e.g. indolizinyl, benzoimidazolyl, coumarinyl, quinolinyl, isoquinolinyl or benzothienyl).

Examples of heteroaryl groups include, but are not limited to, oxazole, pyrrole, imidazole, pyrazole, pyridine, pyrazine, pyrimidine, pyridazine, indolizine, isoindole, indole, indazole, purine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isothiazole, phenoxazine, phenothiazine, thiazole, thiadiazoles, oxadiazole, oxatriazole, tetrazole, thiophene, benzo[b]thiophene, triazole, imidazopyridine and the like.

“Cycloalkyl” refers to cyclic alkyl groups having a single cyclic ring or multiple condensed rings, preferably incorporating 3 to 11 carbon atoms. Such cycloalkyl groups include, by way of example, single ring structures such as cyclopropyl, cyclobutyl, cyclopentyl, cyclohexyl, cyclooctyl, and the like, or multiple ring structures such as adamantanyl, indanyl, 1,2,3,4-tetrahydronapthalenyl and the like.

“Cycloalkenyl” refers to cyclic alkenyl groups having a single cyclic ring or multiple condensed rings, preferably incorporating 3 to 11 carbon atoms. Such cycloalkenyl groups include, by way of example, single ring structures such as cyclopropenyl, cyclobutenyl, cyclopentenyl, cyclohexenyl, cyclooctenyl, and the like, or multiple ring structures such as indenyl, norbornenyl and the like.

“Heterocyclyl” refers to a monovalent saturated or unsaturated group having a single ring or multiple condensed rings, preferably from 1 to 8 carbon atoms and from 1 to 4 hetero atoms selected from nitrogen, sulfur, oxygen, selenium or phosphorous within the ring. The most preferred heteroatom is nitrogen. It will be understood that where, for instance, R₂ or R′ is an optionally substituted heterocyclyl which has one or more ring heteroatoms, the heterocyclyl group can be connected to the core molecule of the compounds of the present invention, through a C—C or C-heteroatom bond, in particular a C—N bond.

Examples of heterocyclyl and heteroaryl groups include, but are not limited to, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isothiazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiadiazoles, oxadiazole, oxatriazole, tetrazole, thiazolidine, thiophene, benzo[b]thiophene, morpholino, piperidinyl, pyrrolidine, tetrahydrofuranyl, triazole, and the like.

“Cyano” refers to the group —C≡N.

When a range of values is specified, for example C₁₋₄ alkyl, it encompasses each value within the range, as well as, all possible intervening ranges. For example, C₁₋₄ alkyl, this includes C₁, C₂, C₃, C₄, C₁₋₄, C₂₋₄, C₃₋₄, C₁₋₃, C₂₋₃, and C₁₋₂ alkyl.

In this specification “optionally substituted” is taken to mean that a group may or may not be further substituted or fused (so as to form a condensed polycyclic group) with one or more groups selected from hydroxyl, acyl, alkyl, alkoxy, alkenyl, alkenyloxy, alkynyl, alkynyloxy, amino, aminoacyl, thio, arylalkyl, arylalkoxy, aryl, aryloxy, carboxyl, acylamino, cyano, halogen, nitro, phosphono, sulfo, phosphorylamino, phosphinyl, heteroaryl, heteroarylalkyl, heteroaryloxy, heterocyclyl, heterocyclylalkyl, heterocyclyloxy, oxyacyl, oxime, oxime ether, hydrazone, oxyacylamino, oxysulfonylamino, aminoacyloxy, trihalomethyl, trialkylsilyl, pentafluoroethyl, trifluoromethoxy, difluoromethoxy, trifluoromethanethio, trifluoroethenyl, mono- and di-alkylamino, mono- and di-(substituted alkyl)amino, mono- and di-arylamino, mono- and di-heteroarylamino, mono- and di-heterocyclyl amino, and unsymmetric di-substituted amines having different substituents selected from alkyl, aryl, heteroaryl and heterocyclyl, and the like, and may also include a bond to a solid support material, (for example, substituted onto a polymer resin). For instance, an “optionally substituted amino” group may include amino acid and peptide residues.

The present disclosure pertains to compounds and their various forms, including ionic forms, tautomers, isomers, polymorphs, pseudopolymorphs, amorphous forms, solvates, co-crystals, chelates, esters, prodrugs, and protected forms. The disclosure also encompasses methods of utilizing these compounds for various purposes. It should be noted that terms like “crystalline form,” “polymorph,” can be used interchangeably to include all crystalline and amorphous forms, such as polymorphs, pseudopolymorphs, solvates (including hydrates), co-crystals, unsolvated polymorphs (including anhydrates), conformational polymorphs, amorphous forms, and mixtures thereof, unless a specific crystalline or amorphous form is specified. In certain embodiments, the compounds and their subgroups include polymorphs, solvates, co-crystals, isomers, tautomers, and/or oxides. In other embodiments, they may include polymorphs, solvates, and/or co-crystals.

“Isomer” includes especially optical isomers (for example essentially pure enantiomers, essentially pure diastereomers, and mixtures thereof) as well as conformation isomers (i.e. isomers that differ only in their angles of at least one chemical bond), position isomers (particularly tautomers), and geometric isomers (e.g. cis-trans isomers).

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. The invention additionally encompasses compounds described herein as individual isomers substantially free of other isomers, and alternatively, as mixtures of various isomers. “Optically-enriched,” as used herein, means that the compound is made up of a significantly greater proportion of one enantiomer. In certain embodiments the compound of the present invention is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972).

Without wanting to be bound by theory, GPR146 is associated with many cardio-metabolic diseases traits, including blood total cholesterol, LDL cholesterol, TG levels; liver inflammation enzyme (AST, GGT, ALP) levels; and hypertension. GPR146 also regulates hypercholesterolemia and liver inflammation in mouse models. It is also suggested that GPR146 regulates pulmonary hypertension. GPR146 promotes activity of hepatic sterol regulatory element binding protein (SREBP2) through extracellular signal-regulated kinase (ERK) signalling pathway upon feeding, thereby regulating hepatic very low-density lipoprotein (VLDL) secretion, and subsequently circulating LDL-C and TG levels. By inhibiting GPR146, cholesterol levels, TG levels, plasma lipid levels may be reduced, leading to therapeutic effects in multiple cardio-metabolic diseases.

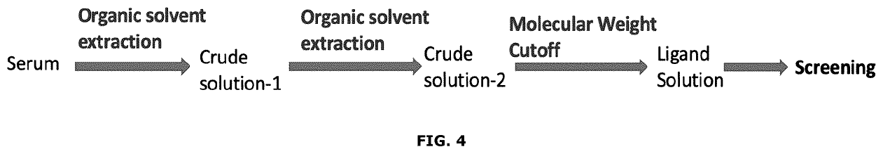

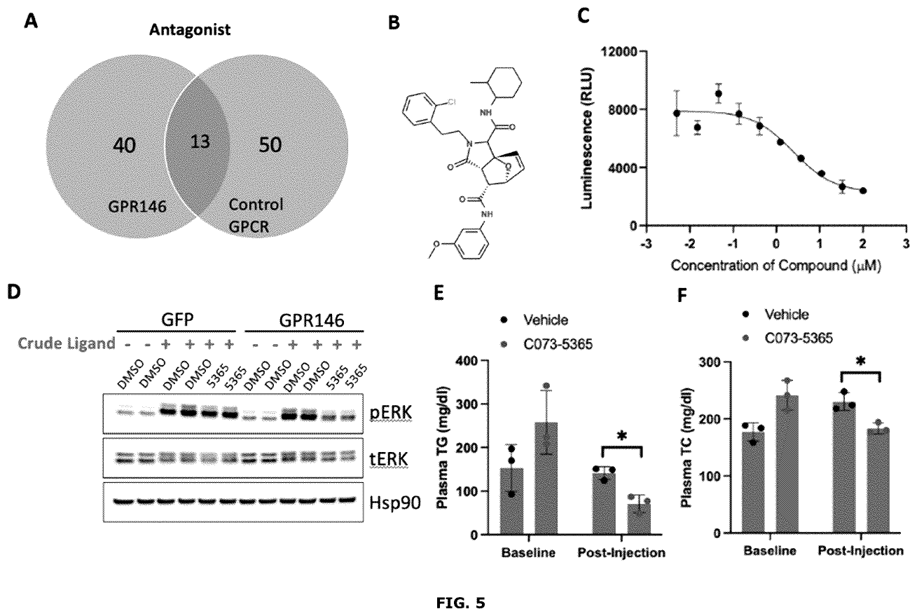

To identify small molecule antagonists inhibiting the bioactivity of GPR146, the inventors developed a crude ligand solution and used it as agonist for the screening of antagonist (FIG. 4). By employing a β-arrestin recruitment assay, the inventors performed the screening with diverse compound libraries in an in-house database. An initial screening of about 25,000 compounds was done. After subsequent counter-screening and cell toxicity screening, the inventors identified 27 compounds as a potential antagonist of GPR146. FIG. 5A shows that 40 compounds were first identified as potential antagonists for GPR146. Out of these, 13 compounds were found to exhibit activity against both GPR146 and control GPCR, indicating that they may not be specific to GPR146. The remaining 27 compounds were found to exhibit activity against GPR146 only and were thus identified as potential GPR146-specific antagonists. In vitro functional validation via β-arrestin recruitment assay showed that the 27 compounds inhibit GPR146 activity.

Accordingly, the present disclosure provides a method of treating a disease and/or condition associated with GPR146.

The present disclosure concerns a method of treating a disease and/or condition associated with GPR146 in a subject in need thereof, comprising administering to the subject a therapeutically effective amount of a compound of Formula (I) or a pharmaceutically acceptable salt, solvate, stereoisomer or prodrug thereof:

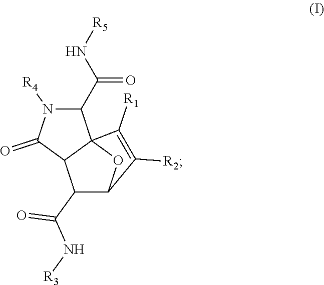

wherein
R₁ is selected from H, halo, cyano, optionally substituted alkyl, and optionally substituted alkenyl;
R₂ is selected from H, halo, cyano, optionally substituted alkyl, and optionally substituted alkenyl;
R₃ is selected from optionally substituted aryl and optionally substituted heteroaryl.
R₄ is selected from optionally substituted alkyl and optionally substituted alkenyl; and
R₅ is selected from optionally substituted cycloalkyl and optionally substituted heterocyclyl.

In some embodiments, R₁ is selected from H, halo, cyano, optionally substituted alkyl, and optionally substituted alkenyl. In some embodiments, R₁ is selected from H, halo, cyano, optionally substituted C₁-C₅ alkyl, and optionally substituted C₁-C₅ alkenyl. In some embodiments, R₁ is H.

In some embodiments, R₂ is selected from H, halo, cyano, optionally substituted alkyl, and optionally substituted alkenyl. In some embodiments, R₂ is selected from H, halo, cyano, optionally substituted C₁-C₅ alkyl, and optionally substituted C₁-C₅ alkenyl. In some embodiments, R₂ is H.

In some embodiments, R₃ is selected from optionally substituted aryl and optionally substituted heteroaryl. In some embodiments, R₃ is aryl optionally substituted with alkoxy. In some embodiments, R₃ is aryl optionally substituted with C₁-C₅ alkoxy. In some embodiments, R₃ is aryl meta substituted with alkoxy. In some embodiments, R₃ is aryl meta substituted with C₁-C₅ alkoxy.

In some embodiments, R₄ is selected from optionally substituted alkyl and optionally substituted alkenyl. In some embodiments, R₄ is optionally substituted alkyl. In some embodiments, R₄ is selected from alkyl substituted with optionally substituted aryl and alkyl substituted with optionally substituted cycloalkenyl. In some embodiments, R₄ is selected from C₁-C₅ alkyl substituted with optionally substituted aryl and C₁-C₅ alkyl substituted with optionally substituted cycloalkenyl. In some embodiments, R₄ is C₁-C₅ alkyl substituted with optionally substituted aryl. In some embodiments, R₄ is C₁-C₅ alkyl substituted with ortho substituted aryl. In some embodiments, the optionally substituent is halo. In some embodiments, the substituent is halo. In some embodiments, the halo is Cl. In some embodiments, R₄ is C₁-C₅ alkyl substituted with optionally substituted cycloalkenyl. In some embodiments, R₄ is C₁-C₅ alkyl substituted with cyclohexenyl.

In some embodiments, R₅ is optionally substituted cycloalkyl and optionally substituted heterocyclyl. In some embodiments, R₅ is optionally substituted cyclohexyl. In some embodiments, R₅ is cyclohexyl optionally substituted with alkyl. In some embodiments, R₅ is cyclohexyl optionally substituted with C₁-C₅ alkyl. In some embodiments, R₅ is cyclohexyl ortho substituted with alkyl. In some embodiments, R₅ is cyclohexyl ortho substituted with C₁-C₅ alkyl.

In some embodiments, the compound of Formula (I) is a compound of Formula (Ia):

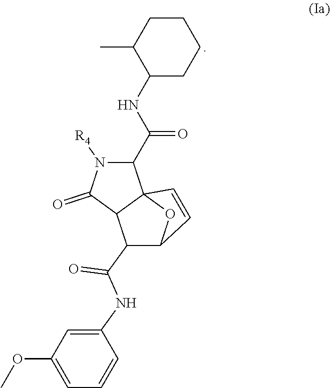

In some embodiments, the compound of Formula (I) is selected from:

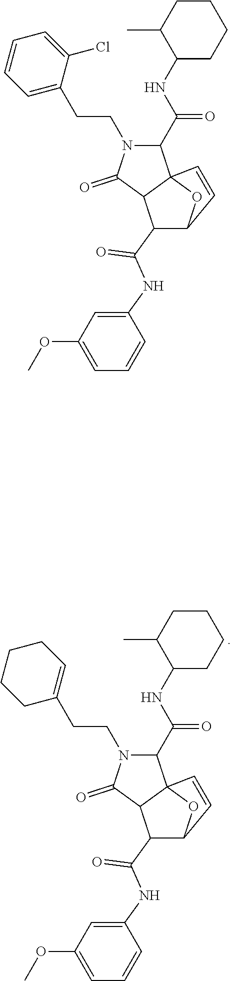 [SMILES: COc1cccc(NC(=O)C2C3C=CC4(O3)C2C(=O)N(CCc2ccccc2Cl)C4C(=O)NC2CCCCC2C)c1 ; COc1cccc(NC(=O)C2C3C=CC4(O3)C2C(=O)N(CCC2=CCCCC2)C4C(=O)NC2CCCCC2C)c1]

In some embodiments, the compound of Formula (I) is a compound of Formula (Ib):

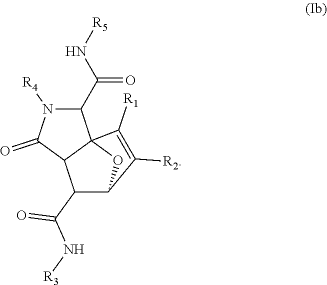

In some embodiments, the compound of Formula (I) is a compound of Formula (Ic):

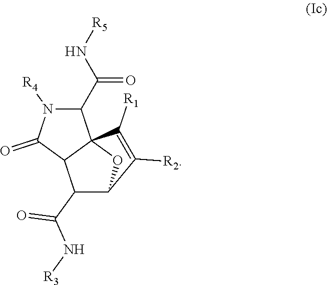

In some embodiments, the compound of Formula (I) is a compound of Formula (Id):

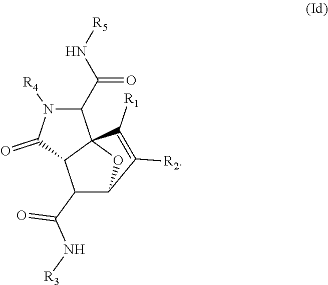

In some embodiments, the compound of Formula (I) is a compound of Formula (Ie):

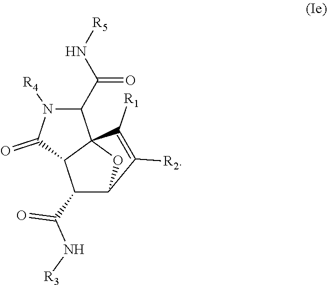

In some embodiments, the compound of Formula (I) is selected from:

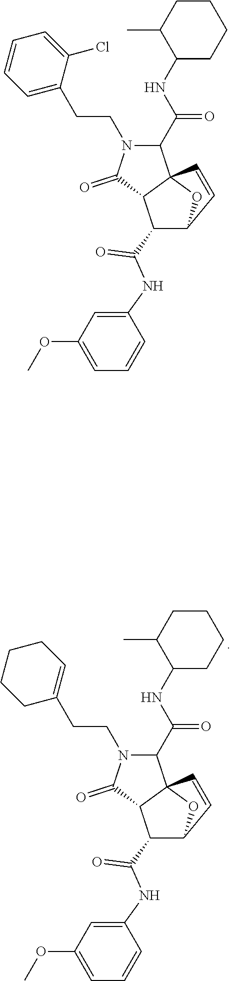

The present disclosure also concerns a compound of Formula (I) or a pharmaceutically acceptable salt, solvate, stereoisomer or prodrug thereof for use in treatment.

The present disclosure also concerns a compound of Formula (I) or a pharmaceutically acceptable salt, solvate, stereoisomer or prodrug thereof for use in treating a disease and/or condition associated with GPR146.

The present disclosure also concerns a use of a compound of Formula (I) or a pharmaceutically acceptable salt, solvate, stereoisomer or prodrug thereof in the manufacture of a medicament for the treatment of a disease and/or condition associated with GPR146.

The present disclosure concerns a method of treating a disease and/or condition associated with GPR146 in a subject in need thereof, comprising administering to the subject a therapeutically effective amount of a compound of Formula (Ia) or a pharmaceutically acceptable salt, solvate, stereoisomer or prodrug thereof:

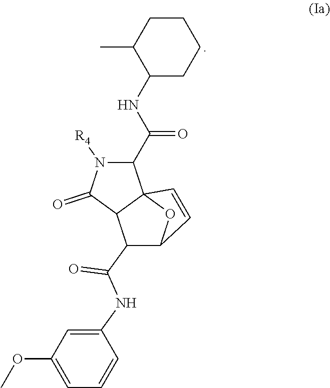

The present disclosure also concerns a compound of Formula (Ia) or a pharmaceutically acceptable salt, solvate, stereoisomer or prodrug thereof for use in treating a disease and/or condition associated with GPR146 in a subject in need thereof.

The present disclosure also concerns a use of a compound of Formula (Ia) or a pharmaceutically acceptable salt, solvate, stereoisomer or prodrug thereof in the manufacture of a medicament for the treatment of a disease and/or condition associated with GPR146.

In some embodiments, the compound of Formula (Ia) is a compound of Formula (If):

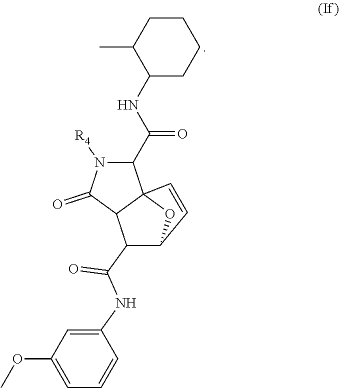

In some embodiments, the compound of Formula (Ia) is a compound of Formula (Ig):

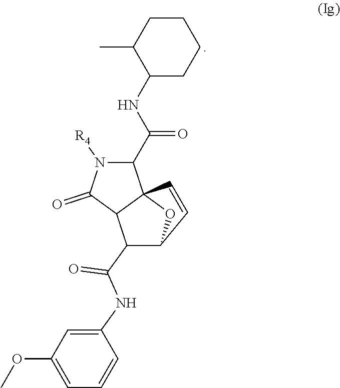

In some embodiments, the compound of Formula (Ia) is a compound of Formula (Ih):

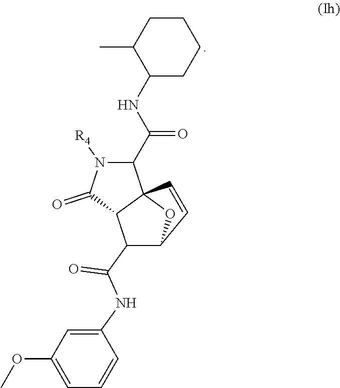

In some embodiments, the compound of Formula (Ia) is a compound of Formula (II):

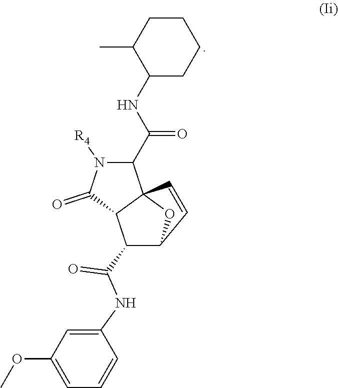

In some embodiments, the compound of Formula (Ia) is selected from:

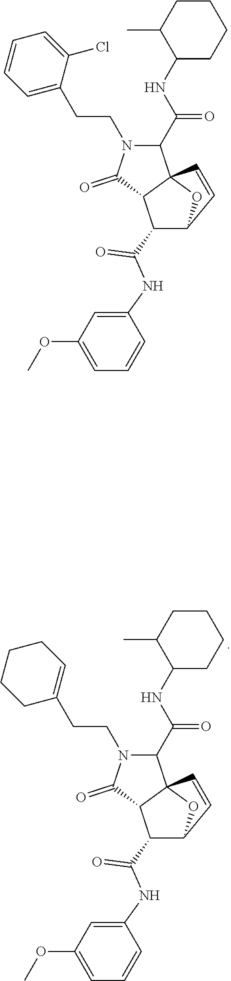

In some embodiments, the disease and/or condition associated with GPR146 is a disease or condition associated with a dysregulation of GPR146. In some embodiments, the disease and/or condition associated with GPR146 is a disease or condition associated with an up-regulation of GPR146. In some embodiments, the disease and/or condition associated with GPR146 is a disease or condition associated with a down-regulation of GPR146.

In some embodiments, the disease and/or condition associated with GPR146 is a cardiometabolic disease. In some embodiments, the disease and/or condition associated with GPR146 is selected from cardiovascular disease, diabetes, kidney disease, liver disease, and a combination thereof. The disease or condition may be hyperlipidedia, hypercholesterolemia, hypertriglyceridemia, atherosclerosis, obesity, fatty liver disease, coronary artery disease, liver inflammation and hypertension.

In some embodiments, the compound of Formula (I) is an antagonist of GPR146. In this regard, the compound inhibits GPR146. The compound may downregulate GPR146.

In some embodiments, the compound of Formula (I) inhibits extracellular signal-regulated kinase (ERK) activation. GPR146 upon ligand binding may promote (ERKs) activation both in vitro and in vivo. Functional validation revealed that the compound of Formula (I) suppressed both receptor activation-stimulated β-arrestin recruitment and downstream ERK signalling activation (FIG. 5B-5D).

In some embodiments, the compound of Formula (I) is characterised by a IC50 of about 1.5 μM to about 3.5 μM. In other embodiments, IC50 is about 1.5 μM to about 3.0 μM, about 1.5 μM to about 2.5 μM, about 2.0 μM to about 3.5 μM, about 2.0 μM to about 3.0 μM, about 2.0 μM to about 2.5 μM, about 2.5 μM to about 3.5 μM, or about 2.5 μM to about 3.0 μM. In some embodiments, the compound of Formula (I) is characterised by a IC50 of about 2.5 μM.

In some embodiments, the compound of Formula (I) is characterised by a reduction in blood total cholesterol (TC) level of about 30% to about 60%. In other embodiments, the reduction is about 30% to about 50%, about 30% to about 40%, about 40% to about 60%, about 40% to about 50%, or about 50% to about 60%. In some embodiments, the reduction is about 50%.

In some embodiments, the compound of Formula (I) is characterised by a reduction in triglyceride (TG) levels of about 15% to about 25%. In other embodiments, the reduction is about 15% to about 23%, about 15% to about 21%, about 18% to about 25%, about 18% to about 23%, about 18% to about 21%, about 21% to about 25%, or about 21% to about 23%. In some embodiments, the reduction is about 21%.

In some embodiments, the compound of Formula (I) is characterised by a cytotoxicity of about 50% to about 95%. In some embodiments, the cytotoxicity is about 50% to about 90%, about 50% to about 85%, about 50% to about 80%, about 50% to about 75%, about 50% to about 70%, about 50% to about 65%, about 50% to about 55%, about 55% to about 95%, about 55% to about 90%, about 55% to about 85%, about 55% toa bout 80%, about 55% to about 75%, about 55% to about 70%, about 55% to about 65%, about 55% to about 60%, about 60% to about 95%, about 60% to about 90%, about 60% to about 85%, about 60% to about 80%, about 60% to about 75%, about 60% to about 70%, about 60% to about 65%, about 70% to about 95%, about 70% to about 90%, about 70% to about 85%, about 70% to about 80%, about 70% to about 75%, about 75% to about 95%, about 75% to about 90%, about 75% to about 85%, or about 75% to about 80%.

The present disclosure also provides a pharmaceutical composition comprising a compound of Formula (I), a compound of Formula (Ia) or a pharmaceutically acceptable salt, solvate or prodrug thereof. The pharmaceutical composition may further comprise a pharmaceutically acceptable excipient.

The compound of the invention can be administered to a subject as a pharmaceutically acceptable salt thereof. Suitable pharmaceutically acceptable salts include, but are not limited to salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

It will be appreciated that any compound that is a prodrug of the compound of formula (I) and/or the compound of Formula (Ia) is also within the scope and spirit of the invention. Thus, the compound of the invention can be administered to a subject in the form of a pharmaceutically acceptable pro-drug. The term “pro-drug” is used in its broadest sense and encompasses those derivatives that are converted in vivo to the compound of the invention. Such derivatives would readily occur to those skilled in the art. Other texts which generally describe prodrugs (and the preparation thereof) include: Design of Prodrugs, 1985, H. Bundgaard (Elsevier); The Practice of Medicinal Chemistry, 1996, Camille G. Wermuth et al., Chapter 31 (Academic Press); and A Textbook of Drug Design and Development, 1991, Bundgaard et al., Chapter 5, (Harwood Academic Publishers).

The compound of the invention may be in crystalline form either as the free compound or as a solvate (e.g. hydrate) and it is intended that both forms are within the scope of the present invention. Methods of solvation are generally known within the art.

The compound of the invention, or a pharmaceutically acceptable salt, solvate or prodrug thereof is administered to the patient in a therapeutically effective amount. As used herein, a therapeutically effective amount is intended to include at least partially attaining the desired effect, or delaying the onset of, or inhibiting the progression of, or halting or reversing altogether the onset or progression of macular degeneration.

The term “therapeutic effect” refers to some extent of relief of one or more of the symptoms of a disorder (e.g., a neoplasia or tumour) or its associated pathology. “Therapeutically effective amount” as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds of the invention employed in a pharmaceutical composition at levels lower than that required in order to achieve the desired therapeutic effect and gradually increase the dosage until the desired effect is achieved.

As used herein, the term “effective amount” relates to an amount of compound which, when administered according to a desired dosing regimen, provides the desired therapeutic activity. Dosing may occur at intervals of minutes, hours, days, weeks, months or years or continuously over any one of these periods. Suitable dosages may lie within the range of about 0.1 ng per kg of body weight to 1 g per kg of body weight per dosage, such as is in the range of 1 mg to 1 g per kg of body weight per dosage. In one embodiment, the dosage may be in the range of 1 mg to 1000 mg per kg of body weight per dosage. In another embodiment, the dosage may be in the range of 1 mg to 800 mg per kg of body weight per dosage. In yet another embodiment, the dosage may be in the range of 1 mg to 500 mg per kg of body weight per dosage, such as up to 250 mg per body weight per dosage.

Suitable dosage amounts and dosing regimens can be determined by the attending physician and may depend on the severity of the condition as well as the general age, health and weight of the patient to be treated.

The compound of the invention may be administered in a single dose or a series of doses. While it is possible for the active ingredient to be administered alone, it is preferable to present it as a composition, preferably as a pharmaceutical composition. The formulation of such compositions is well known to those skilled in the art. The composition may contain any suitable carriers, diluents or excipients. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

The carrier must be pharmaceutically “acceptable” in the sense of being compatible with the other ingredients of the composition and not injurious to the patient. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

Modes of administration including topical or intravenous administration may be possible. For example, solutions or suspensions of the compound, composition or combinations of the invention may be formulated as eye drops, or as a membranous ocular patch, which is applied directly to the surface of the eye. Topical application typically involves administering the compound of the invention in an amount between 0.1 ng and 10 mg.

The compound or composition of the invention may also be suitable for intravenous administration. For example, a compound of Formula (I) or a pharmaceutically acceptable salt, solvate or prodrug thereof may be administered intravenously at a dose of up to 100 mg/m².

The compound or composition of the invention may also be suitable for oral administration and may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste. In another embodiment, the compound of Formula (I) or a pharmaceutically acceptable salt, solvate or prodrug is orally administrable.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. inert diluent, preservative disintegrant (e.g. sodium starch glycolate, cross-linked polyvinyl pyrrolidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

The compound or composition of the invention may be suitable for topical administration in the mouth including lozenges comprising the active ingredient in a flavoured base, usually sucrose and acacia or tragacanth gum; pastilles comprising the active ingredient in an inert basis such as gelatine and glycerin, or sucrose and acacia gum; and mouthwashes comprising the active ingredient in a suitable liquid carrier.

The compound or composition of the invention may be suitable for parenteral administration include aqueous and non-aqueous isotonic sterile injection solutions which may contain anti-oxidants, buffers, bactericides and solutes which render the compound, composition or combination isotonic with the blood of the intended recipient; and aqueous and non-aqueous sterile suspensions which may include suspending agents and thickening agents. The compound, composition or combination may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage composition or combinations are those containing a daily dose or unit, daily sub-dose, as herein above described, or an appropriate fraction thereof, of the active ingredient.

It should be understood that in addition to the active ingredients particularly mentioned above, the composition or combination of this invention may include other agents conventional in the art having regard to the type of composition or combination in question, for example, those suitable for oral administration may include such further agents as binders, sweeteners, thickeners, flavouring agents disintegrating agents, coating agents, preservatives, lubricants and/or time delay agents. Suitable sweeteners include sucrose, lactose, glucose, aspartame or saccharine. Suitable disintegrating agents include cornstarch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

### EXAMPLES

G-protein-coupled receptors (GPCRs) are targets for nearly 40% of all prescription drugs on the market and orphan GPCRs currently have become a primary focus for drug development. Orphan GPR146 is a promising drug target for hyperlipidedia, coronary artery disease, liver inflammation, and hypertension. Human genetic studies have uncovered that GPR146 is significantly associated with many cardio-metabolic diseases traits, including blood total cholesterol, LDL cholesterol, TG levels; liver inflammation enzyme (AST, GGT, ALP) levels; and hypertension. In line with human genetic studies, my previous functional study discovered that GPR146 regulates hypercholesterolemia and liver inflammation in mouse models. Inhibition of GPR146 in mouse model of homozygous familial hypercholesterolemia resulted in 55% and 70% reductions in plasma cholesterol and triglycerides levels, respectively, which was accompanied with 90% reduction in atherosclerotic lesion area. Moreover, we discovered that depletion of GPR146 in mice led to 45% reduction in whole body fat mass, as well as 50% reduction in fat levels in liver compared with control mice (unpublished data). Recently, studies from other groups suggested that GPR146 regulates pulmonary hypertension.

To develop therapies targeting GPR146, we have established a robust pipeline to discover small molecule antagonists inhibiting the bioactivity of GPR146. Briefly, we first developed a crude ligand solution and used it as agonist for the screening of antagonist. Next, we established an industry-grade high-throughput screening assay and performed the screening with diverse compound libraries in EDDC, ASTAR.

Through the initial screening of 25 k compounds, we have identified a compound C073-5365 (FIG. 5B) as a potent antagonist of GPR146. In vitro functional validation via β-arrestin recruitment assay showed that the compound inhibits GPR146 activity with a IC50 of 2.468 μM (FIG. 5C). The β-arrestin recruitment assay is a technique used to study the interaction between G protein-coupled receptors (GPCRs) and the intracellular signalling protein β-arrestin. Moreover, GPR146 upon ligand binding promotes ERK activation both in vitro and in vivo. Pre-treatment with the compound potently inhibited ERK activation in HEK293T cells with overexpression of human GPR146. Importantly, functional validation in mouse model of homozygous hypercholesterolemia (Ldlr−/−) revealed that mice upon injection with a dose of 2.5 mg/kg C073-5365 showed a 50% and 21% reductions in blood TG and TC levels, respectively, compared to control littermates receiving vehicle. Taken together, our data has demonstrated that C073-5365 is an antagonist of orphan GPR146.

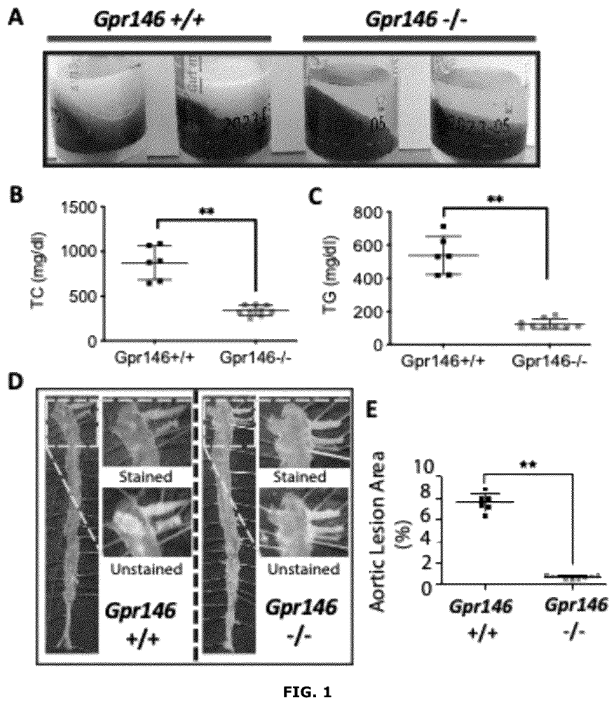

Our studies in mice showed that GPR146 promotes activity of hepatic sterol regulatory element binding protein (SREBP2) through extracellular signal-regulated kinase (ERK) signalling pathway upon feeding, thereby regulating hepatic very low-density lipoprotein (VLDL) secretion, and subsequently circulating LDL-C and TG levels. We found that the LDL lowering effect upon GPR146 depletion does not require intact LDLR. Depletion of GPR146 in mice lacking LDLR led to ˜60% and ˜77% reductions of plasma TC and TG levels, respectively, in male mice fed with western diet (FIG. 1A-1C). In line with substantially reduced plasma lipid levels, atherosclerotic lesion area evaluated by en face Oil Red O staining of the full-length aorta was drastically reduced by ˜90% in male Gpr146−/− Ldlr−/− mice fed WD for 16 weeks compared to Gpr146+/+ Ldlr−/− littermates (FIGS. 1D and 1E).

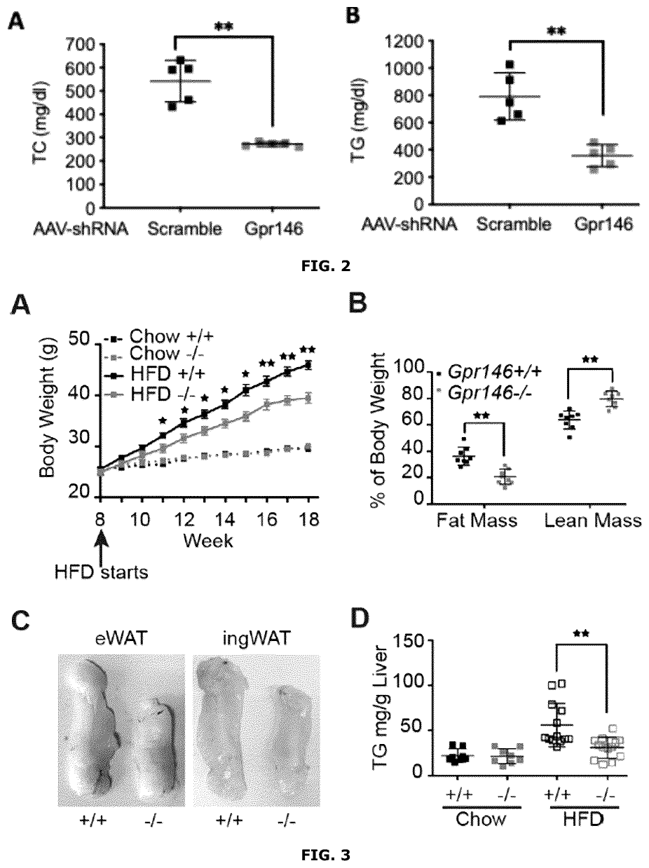

The robust lipid-lowering and concomitant atherosclerosis-protective effects upon GPR146 germline knockout might be due to developmental defect caused by the absence of functional GPR146. To gain more direct evidence demonstrating that acute inhibition of GPR146 would be sufficient to reduce plasma lipid levels in adult mice, we injected AAV8-shRNA to deplete hepatic GPR146 in adult Ldlr−/− mice and found that plasma TC and TG levels were reduced by ˜55% and ˜55%, respectively, in male GPR146-depleted mice compared to control littermates upon four weeks of WD feeding (FIGS. 2A and 2B). These data suggest that acute depletion of GPR146 in liver of adult mice reduced plasma lipid levels to a similar extent compared to germline knockout of GPR146.

Moreover, we found that depletion of GPR146 led to reduced body weight upon high-fat diet (HFD) feeding compared to wild-type littermates (FIG. 3A). Magnetic resonance imaging (MRI) analysis showed that Gpr146−/− mice exhibited about 45% reduction of fat mass compared to Gpr146+/+ littermates upon HFD feeding (FIG. 3B). In addition, we found that depletion of Gpr146 led to reduced liver TG levels upon high-fat diet (HFD) feeding compared to wild-type littermates (FIG. 3D).

### GPR146 Antagonists

To develop small molecule antagonists inhibiting GPR146, we prepared crude ligand solution and used it as tool agonist for the screening of antagonists (FIG. 4). By employing a β-arrestin recruitment assay (PathHunter, DiscoverX), we screened 25 k diverse compounds stored in our database. After subsequent counter-screening and cell toxicity screening, we have identified 27 candidate antagonists for GPR146 (FIG. 5A). Further functional validation of 10 top candidates revealed that Compound of Formula (Ia) robustly suppressed both receptor activation-stimulated β-arrestin recruitment and downstream ERK signalling activation (FIG. 5B-5D).

To test the biological effects of Compound of Formula (Ia) in vivo, Ldlr−/− mice were administrated by intraperitoneal injection (2.5 mg/kg body weight) for two consecutive days followed by analysis of blood cholesterol and TG levels. Mice upon Compound of Formula (I) administration exhibited significantly reduced blood total cholesterol and TG levels by 50% and 21%, respectively, compared to control littermates receiving vehicle (FIGS. 5E and 5F). Taken together, these data suggest that Compound of Formula (I) potently inhibits GPR146 signalling both in vitro and in vivo. This compound may be useful for treating hyperlipidemia and other diseases regulated by GPR146.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

What is claimed is:

1. A method of treating a disease and/or condition associated with GPR146 in a subject in need thereof, comprising administering to the subject a therapeutically effective amount of a compound of Formula (I) or a pharmaceutically acceptable salt, solvate, stereoisomer or prodrug thereof: 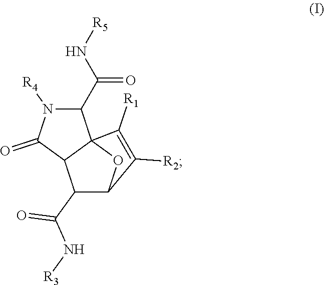
wherein
R₁ is H, halo, cyano, optionally substituted alkyl, or optionally substituted alkenyl;
R₂ is H, halo, cyano, optionally substituted alkyl, or optionally substituted alkenyl;
R₃ is optionally substituted aryl or optionally substituted heteroaryl;
R₄ is optionally substituted alkyl or optionally substituted alkenyl; and
R₅ is optionally substituted cycloalkyl or optionally substituted heterocyclyl.

2. The method according to claim 1, wherein R₁ is H.

3. The method according to claim 1, wherein R₂ is H.

4. The method according to claim 1, wherein R₃ is aryl optionally substituted with alkoxy.

5. The method according to claim 1, wherein R₄ is C₁-C₅ alkyl substituted with optionally substituted aryl or C₁-C₅ alkyl substituted with optionally substituted cycloalkenyl.

6. The method according to claim 1, wherein R₅ is cyclohexyl optionally substituted with alkyl.

7. The method according to claim 1, wherein the compound of Formula (I) is a compound of Formula (Ia):

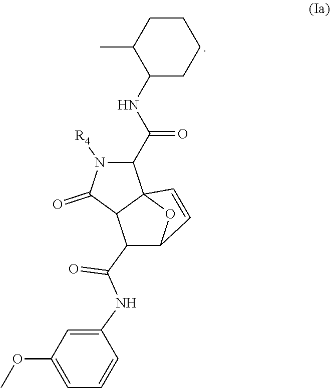

8. The method according to claim 1, wherein the compound of Formula (I) is

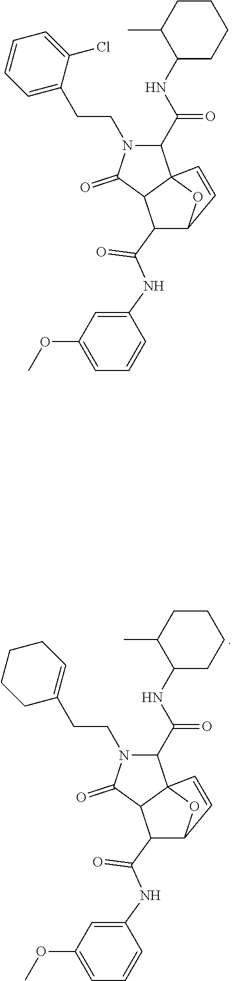 [SMILES: COc1cccc(NC(=O)C2C3C=CC4(O3)C2C(=O)N(CCc2ccccc2Cl)C4C(=O)NC2CCCCC2C)c1 ; COc1cccc(NC(=O)C2C3C=CC4(O3)C2C(=O)N(CCC2=CCCCC2)C4C(=O)NC2CCCCC2C)c1]

9. A method of treating a disease or condition associated with GPR146 in a subject in need thereof, comprising administering to the subject a therapeutically effective amount of a compound of Formula (Ia) or a pharmaceutically acceptable salt, solvate, stereoisomer or prodrug thereof:

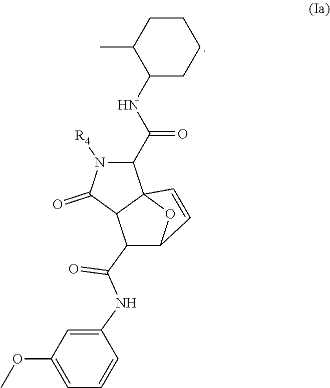

10. The method according to claim 1, wherein the disease or condition associated with GPR146 is a disease and/or condition associated with a dysregulation of GPR146.

11. The method according to claim 1, wherein the disease and/or condition associated with GPR146 is a cardiometabolic disease.

12. The method according to claim 1, wherein the disease and/or condition associated with GPR146 is cardiovascular disease, diabetes, kidney disease, liver disease, or a combination thereof.

13. The method according to claim 1, wherein the disease and/or condition associated with GPR146 is hyperlipidedia, hypercholesterolemia, hypertriglyceridemia, atherosclerosis, obesity, fatty liver disease, coronary artery disease, liver inflammation or hypertension.

14. The method according to claim 1, wherein the compound of Formula (I) inhibits GPR146.

15. The method according to claim 1, wherein the compound of Formula (I) inhibits extracellular signal-regulated kinase (ERK) activation.

16. The method according to claim 1, wherein the compound of Formula (I) is characterised by a IC50 of about 1.5 μM to about 3.5 μM.

17. The method according to claim 1, wherein the compound of Formula (I) is characterised by a reduction in blood total cholesterol level of about 30% to about 60%.

18. The method according to claim 1, wherein the compound of Formula (I) is characterised by a reduction in triglyceride levels of about 15% to about 25%.

19. The method according to claim 1, wherein the compound of Formula (I) is characterised by a cytotoxicity of about 50% to about 95%.

20. A pharmaceutical composition comprising a compound of Formula (I), a compound of Formula (Ia) or a pharmaceutically acceptable salt, solvate, stereoisomer or prodrug thereof; wherein the compound of Formula (I) is: 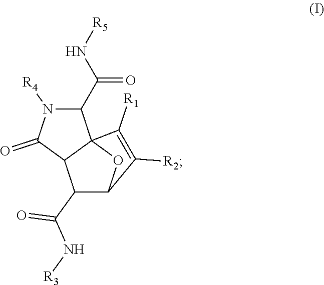
wherein
R₁ is H, halo, cyano, optionally substituted alkyl, or optionally substituted alkenyl;
R₂ is H, halo, cyano, optionally substituted alkyl, or optionally substituted alkenyl;
R₃ is optionally substituted aryl or optionally substituted heteroaryl;
R₄ is optionally substituted alkyl or optionally substituted alkenyl; and
R₅ is optionally substituted cycloalkyl or optionally substituted heterocycyl; and
wherein the compound of Formula (Ia) is: [chemical structure]
